(19) United States
(12) Patent Application Publication
(10) Pub. No.: US 2024/0427421 A1
(43) Pub. Date: Dec. 26, 2024

(54) Pneumatic Sensing and Control System for Sleep Devices

(71) Applicant: Apple lnc., Cupertino, CA (US)

(72) Inventors: Wegene H. Tadele, San Francisco, CA (US); Yingjun Zhang, Shanghai (CN); Siddharth Nangia, San Francisco, CA (US)

(21) Appl. No.: 18/687,201

(22) PCT Filed: Aug. 31, 2022
(86) PCT No.: PCT/CN2022/116026

§ 371 Date: Feb. 27, 2024

Related U.S. Application Data
(60) Provisional application No. 63/239,241, filed on Aug. 31, 2021.

(51) Int. Cl. G06F 3/01; G06F 3/0488

(52) U.S. Cl. CPC G06F 3/016; G06F 3/0488; G06F 2203/04105

(57) ABSTRACT

Embodiments are directed to in-bed haptic systems including a pneumatic pad configured for placement between a user and a bed. The pneumatic pad can include an array of actuation cells, where each actuation cell in the array of actuation cells is configured to actuate in response to fluid being introduced into the actuation cell. The system can include an input device that includes a touch sensitive display and is configured to display a set of haptic sequences that can be applied to the array of actuation cells in a user interface of the input device and receive an input at the touch sensitive display of a selected haptic sequence. The system can include a control system that receives an indication of the selected haptic sequence and actuates actuation cells of the array of actuation cells using the selected haptic sequence and the pressure measurements.

### CROSS-REFERENCE TO RELATED APPLICATION

This application claims priority to U.S. Provisional Patent Application No. 63/239,241, filed Aug. 31, 2021, the contents of which are incorporated herein by reference as if fully disclosed herein.

### FIELD

The described embodiments generally relate to bed sensors and/or haptics and, more particularly, to control of on-bed pneumatic sensors and/or pneumatic haptics or systems that otherwise include pneumatic sensors and/or pneumatic haptics.

### BACKGROUND

A variety of sensors may be used to monitor a user's sleep or health while the user is in bed (e.g., while the user is resting or sleeping). A variety of sensors may be similarly used to monitor a user's sleep or health while the user is resting or sleeping on other surfaces.

### SUMMARY

Embodiments are directed to an in-bed haptic system that includes a pneumatic pad configured for placement between a user and a bed. The pneumatic pad can include an array of actuation cells, where each actuation cell in the array of actuation cells is configured to actuate in response to fluid being introduced into the actuation cell and an array of pressure sensors, each pressure sensor in the array of pressure sensors configured to output pressure measurements corresponding to the array of actuation cells. The in-bed haptic system can include an input device comprising a touch sensitive display and configured to display a set of haptic sequences that can be applied to the array of actuation cells in a user interface of the input device and receive an input at the touch sensitive display of a selected haptic sequence of the set of haptic sequences. The in-bed system can also include a control system configured to receive an indication of the selected haptic sequence, receive the pressure measurements, and actuate actuation cells of the array of actuation cells using the selected haptic sequence and the pressure measurements.

Embodiments are also directed to a pneumatic actuation device including a pneumatic pad configured for placement between a user and a bed and comprising an array of actuation cells, where each actuation cell in the array of actuation cells is configured to actuate in response to fluid being introduced into the actuation cell. The pneumatic activation device can include a control system configured to receive a haptic control sequence from a user input to an input device. The haptic control sequence can be generated using the user input to a user interface comprising one or more controls for defining haptic output at the pneumatic pad. The control system can be configured to actuate the array of actuation cells using the haptic control sequence.

Embodiments are further directed to an in-bed haptic system that includes a pneumatic pad configured for placement between a user and a bed and comprising an array of inflatable cells. The haptic actuation system can include an input device configured to display a user interface for defining a haptic output and in response to receiving an input to the user interface, generate a haptic control sequence for applying to the array of inflatable cells. The in-bed haptic system can include a control system configured to receive the haptic control sequence and actuate the pneumatic pad using the haptic control sequence.

### BRIEF DESCRIPTION OF THE DRAWINGS

The disclosure will be readily understood by the following detailed description in conjunction with the accompanying drawings, wherein like reference numerals designate like structural elements, and in which:

FIG. 1 shows an example environment for using an in-bed haptic device;

FIG. 2A shows the example in-bed haptic device and the control system of FIG. 1;

FIGS. 2B-2C show an example haptic output provided by the in-bed haptic device of FIG. 1;

FIGS. 3A-3C show an example haptic output provided by the in-bed haptic device of FIG. 1;

FIG. 4 shows an example block diagram of a control system that is fluidly coupled to an in-bed haptic device;

FIG. 5 shows an example process for operating a haptic system as described herein;

FIGS. 6A and 6B show example user interfaces for defining and/or selecting a haptic actuation sequence to be applied by the actuation cells;

FIG. 7 shows an example user interface for selecting a region of an actuation array for applying a haptic actuation sequence;

FIG. 8 shows an example user interface for selecting anatomical features for applying a haptic actuation sequence; and

FIG. 9 is an example block diagram of an electronic device.

The use of cross-hatching or shading in the accompanying figures is generally provided to clarify the boundaries between adjacent elements and also to facilitate legibility of the figures. Accordingly, neither the presence nor the absence of cross-hatching or shading conveys or indicates any preference or requirement for particular materials, material properties, element proportions, element dimensions, commonalities of similarly illustrated elements, or any other characteristic, attribute, or property for any element illustrated in the accompanying figures.

Additionally, it should be understood that the proportions and dimensions (either relative or absolute) of the various features and elements (and collections and groupings thereof) and the boundaries, separations, and positional relationships presented therebetween, are provided in the accompanying figures merely to facilitate an understanding of the various embodiments described herein and, accordingly, may not necessarily be presented or illustrated to scale, and are not intended to indicate any preference or requirement for an illustrated embodiment to the exclusion of embodiments described with reference thereto.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

Embodiments described herein are directed to on-bed haptic systems that can apply haptic outputs to a user positioned on the bed. The on-bed haptic actuation system can include a pad that includes an array of actuation cells and one or more sensors that may be used to measure parameters of the array of actuation cells and/or physiological parameters of a user positioned on the pad. In some cases, the array of actuation cells can include pneumatic actuation cells that expand in response to fluid being introduced into the cells. Each cell may include a valve and the actuation of each cell may be independently controlled using a respective valve. In some cases, the actuation system can include pressure sensors, which can be used to sense a pressure in one or more of the actuation cells and the pressure measurements can be used to control actuation of the array of actuation cells. In some cases, the pressure in each actuation cell can be independently measured and used to control the air pressure within a respective actuation cell. In other cases, the system can actuate cells without measuring pressures. For example, the system can be initially calibrated and then operate in an open loop mode where actuation cells are inflated based on calibration parameters.

The array of actuation cells can be actuated in various sequences to provide different haptic effects to a user. The actuation sequences can be applied to create a waveform, pattern, or other coordinated output in which the actuation of different actuation cells is independently controlled to create dynamic haptic effects. In some cases, the actuation sequences can define a waveform or pattern such as a wave pattern across the array of actuation cells. The actuation sequences can be configured to produce different types of effects, which can include effects that help a user sleep, provide a wake experience, aid relaxation, relieve stress, or provide other haptic experiences to a user positioned on the system.

The haptic systems can have an input interface that includes options for a user to select and/or define haptic actuation sequences for the array of actuation cells. The input interface can be output by an electronic device such as a smartphone, tablet, computer, wearable electronic device, or any other suitable device. In some cases, the input interface can identify a set of predefined actuation sequences that can be selected by the user. The user may be able to define or adjust parameters of the actuation sequences such as setting an actuation speed, actuation intensity, defining a portion of the array of actuation cells for applying the sequence, identifying an anatomical feature for applying the actuation sequence, and so on. In other cases, the input interface may allow the user to define custom actuation sequences and define one or more parameters for the actuation sequence. For example, the control interface may include an image of the array of actuation cells and the user can define an actuation sequence by interacting with the displayed image of the actuation cells using a touch sensitive display.

The input system may generate a haptic actuation control sequence based on the user inputs and send the generated haptic actuation control sequence to a control system for the array of actuation cells. The control system may use the received haptic actuation control sequence to control actuation of actuation cells of the array of actuation cells to provide a coordinated haptic output. For example, the haptic actuation control sequence may define time-varying actuation profiles for the array of actuation cells and the control system may use the control sequence to generate control signals for controlling actuation of cells of the actuation array. The control system may control actuation of individual pneumatic cells by generating control signals for valves associated with each cell. Accordingly, the haptic system may use user selected and/or defined haptic actuation sequences to output a coordinate haptic effect at an array of actuation cells.

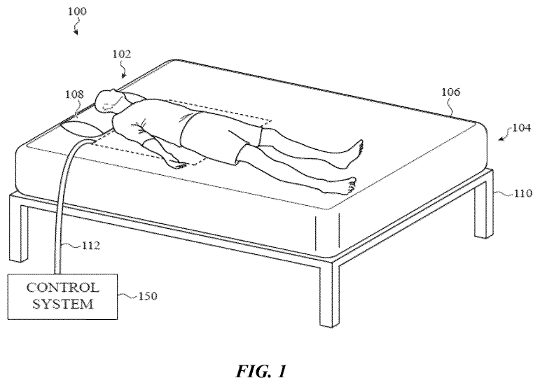

These and other embodiments are discussed below with reference to FIGS. 1-9. However, those skilled in the art will readily appreciate that the detailed description given herein with respect to these Figures is for explanatory purposes only and should not be construed as limiting.

FIG. 1 shows an example in-bed haptic device 100 (shown in phantom). As shown in FIG. 1, the in-bed haptic device 100 may be positioned beneath a user 102 as the user is in a bed 104. The in-bed haptic device 100 may be adapted to provide haptic outputs to users. The haptic outputs provided by the in-bed haptic device 100 may be provided in response to detected inputs at the in-bed haptic device or another electronic device, or in association with notifications, alerts, or other outputs at the in-bed haptic device or another electronic device.

In some cases, the in-bed haptic device 100 is adapted to be positioned between a user 102 and a mattress 106 of a bed 104. The in-bed haptic device 100 may be sufficiently thin and/or flexible so that the in-bed haptic device, when positioned in a bed 104 beneath a user 102, does not cause discomfort. In some cases, a thickness of the in-bed haptic device 100 is much smaller than its length and/or width. For example, the thickness (e.g., a distance between a top surface and a bottom surface) of the in-bed haptic device 100 may be less than approximately ten percent, five percent, or even one percent of the width of the in-bed haptic device. The dimensions of the in-bed haptic device 100 may provide numerous advantages, including increasing a flexibility of the in-bed haptic device, improving comfort of the in-bed haptic device, and/or reducing a user-perceptibility of the in-bed haptic device during use.

The in-bed haptic device 100 may include an array of actuation cells configured to expand and/or contract to provide haptic outputs and/or portions thereof. Actuation cells of the array of actuation cells may be configured to actuate (e.g., expand, contract, or otherwise change shape) in a predetermined sequence to provide haptic outputs. In some cases, the actuation cells include one or more bladders configured to inflate and/or deflate to actuate the actuation cells. The in-bed haptic device 100 may include an enclosure that is configured to be placed beneath a user 102 during use. The in-bed haptic device 100 may provide haptic outputs along a top external surface of the enclosure that may be perceived tactilely by the user 102. In some cases, the top external surface of the enclosure defines a modifiable contour, and actuation of the actuation cells modifies the modifiable contour to provide haptic outputs.

The in-bed haptic device 100 can include an array of sensors that are configured to detect one or more physiological parameters of the user 102. The array of sensors can include pressure sensors that can be used to generate a pressure map of the user, which can be used to determine a profile, position, posture, or other parameters of the user. In some cases, the sensors can be integrated with the actuation cells to measure a fluid pressure within each actuation cell. Additionally or alternatively, the array of sensors can include other types of sensors, such as temperature sensors, proximity sensors, capacitive sensors, piezo-electric sensors, strain-based sensors, acoustic sensors, and vibration sensors, among others.

In various embodiments, the in-bed haptic device 100 and/or the control system 150 may be connected to a companion device configured to provide triggers for providing haptic outputs, control signals, and other information. The companion device may be any suitable electronic device, including a sleep monitor, a wearable electronic device, a timekeeping device, a health monitoring or fitness device, a portable computing device, a mobile phone (including a smart phone), a tablet computing device, a digital media player, a virtual reality device, an audio device (including earbuds and headphones), and the like.

In some cases, the haptic outputs may correspond to inputs, outputs, alerts, or notifications at the in-bed haptic device or another electronic device. As another example, a haptic output may correspond to an alert or notification received at the in-bed haptic device 100 or a connected device, such as a phone call, a received message, a push notification, or the like. In some cases, an alert may correspond to a biometric or similar characteristic of the user 102. For example, the haptic output may be provided in response to a heart rate, breathing rate, or other biometric, detected by the in-bed haptic device 100 or another device, rising above or falling below a predetermined threshold.

In various embodiments, the haptic outputs may be provided while a user is awake or asleep. In some cases, the in-bed haptic device 100 or another device may detect whether a user is awake or asleep and may provide, modify, or cease a haptic output in response to the determination. In some embodiments, the in-bed haptic device 100 may be used to urge a user toward, or keep a user in, a sleep state or a wake state.

The in-bed haptic device 100 may be positioned above or beneath a mattress 106 and/or bed frame 110 of the bed 104. The in-bed haptic device 100 may be positioned above or beneath bedding of the bed 104, including a mattress protector, sheets, blankets, and the like. In some cases, the in-bed haptic device 100 is positioned above the mattress 106 and beneath at least some layers of bedding. For example, the in-bed haptic device 100 may be positioned above a mattress protector, but beneath a bottom sheet of the bedding. In some cases, the in-bed haptic device 100 includes adhesive or a high-friction material along one or more surfaces so that the in-bed haptic device 100 may be attached or coupled to the mattress 106 or bedding of the bed (e.g., a mattress protector). In some cases, the in-bed haptic device 100 is placed between approximately 10 and 40 centimeters from a pillow 108. The in-bed haptic device 100 may be centered in a sleeping area of the user 102.

Although the in-bed haptic device 100 is primarily described in relation to the bed 104, it will be appreciated that the devices described herein can be implemented in a variety of other use cases. For example, the devices described herein can be configured for use on other furniture, cars, hospital beds, cribs, or other suitable structures, which may be generally referred to herein as “user support structures.” The haptic device can be used to determine a pressure map of a user who is sitting on or otherwise engaged with various user support structures. As described herein, the pressure map can be used to identify a particular user, or determine a posture, position, or other parameter of a user. In some cases, the pressure map data can be used to provide feedback to the user, which can include providing alerts or suggestions to the user about their posture or position, providing haptic feedback to the user, and/or providing other alerts or feedback to the user. In some cases, the identity of a user, based on a pressure map, can be used to control a support structure that the user is engaged with, such as adjusting a car seat, adjusting a hospital bed, or other user support structure.

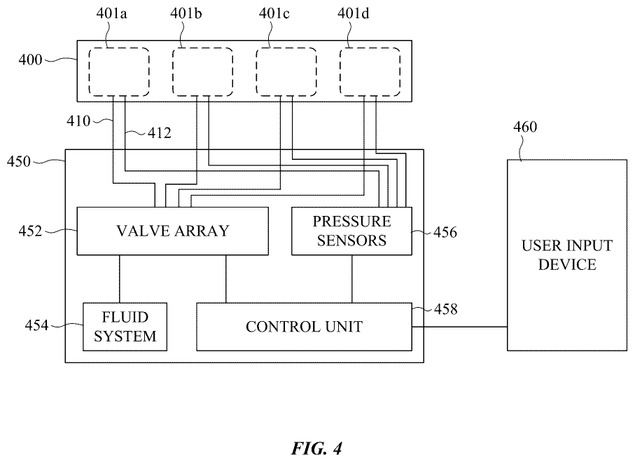

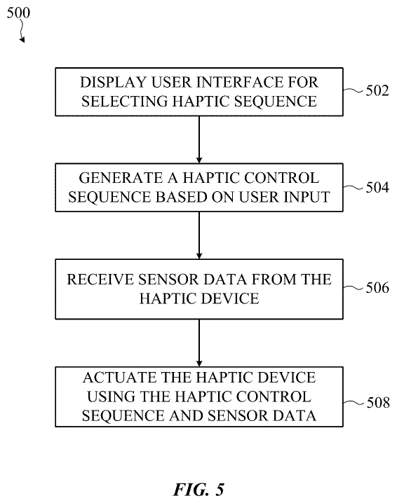

The in-bed haptic device 100 may be operably and/or fluidly coupled to a control system 150. The control system 150 may be configured to introduce pressurized air into one or more actuation cells (e.g., into an interior volume of the bladder(s)) of the in-bed haptic device 100 and/or remove pressurized air from one or more actuation cells (e.g., from an interior volume of the bladder(s)) of the in-bed haptic device in a predetermined sequence to provide haptic outputs. As discussed in more detail below with respect to FIG. 4, the control system 150 may cause haptic outputs to be provided by the in-bed haptic device 100 in response to receiving signals from a processing unit (e.g., a processing unit of the control system 150 or a processing unit of another electronic device).

The control system 150 and/or the in-bed haptic device 100 may include one or more connectors 112 that fluidly couple the control system to the in-bed haptic device. The connector(s) 112 of the control system 150 allow the control system and the in-bed haptic device 100 to be positioned separately from one another. In some cases, the control system 150 may be located far enough away from the in-bed haptic device 100 (and the user 102), such as in another room, that potential disturbances (e.g., sounds, vibrations, and the like) produced by the control system 150 may not disturb the user 102. The connectors 112 can also couple the array of sensors to the in-bed haptic device to the control system 150. In some cases, the array of sensors can be configured to wirelessly transmit sensor data to the control system 150 and/or other electronic device, such as a companion device described herein.

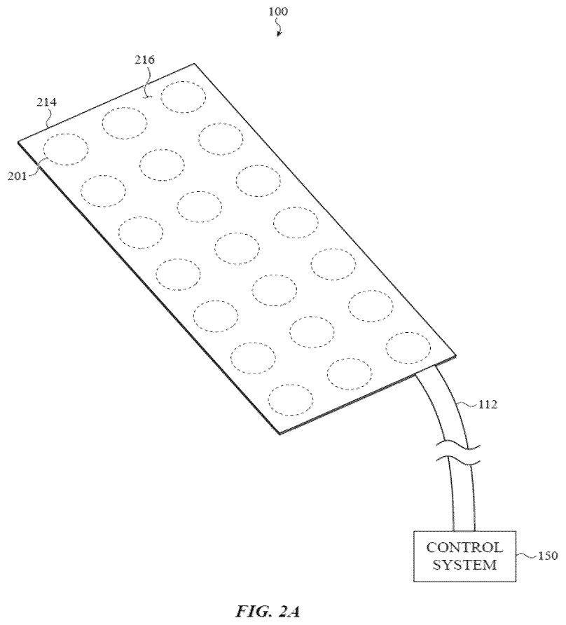

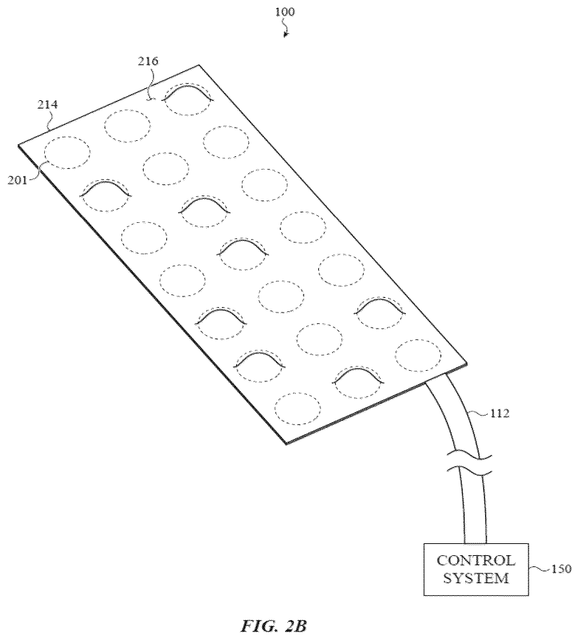

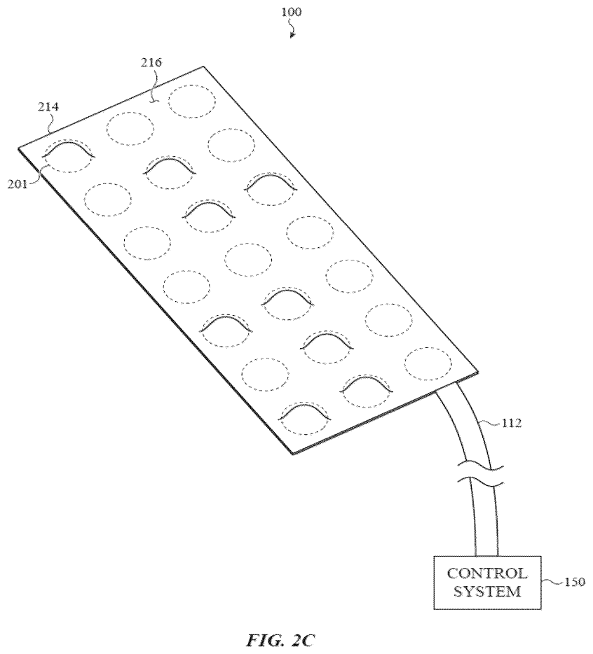

FIG. 2A shows the in-bed haptic device 100 and the control system 150 of FIG. 1. As noted above, the in-bed haptic device 100 may include an array of actuation cells 201 (shown in phantom in FIG. 2A) configured to actuate (e.g., expand, contract, or otherwise change shape) to provide haptic outputs and/or portions thereof. The in-bed haptic device 100 may include an enclosure 214 or other external layer that at least partially surrounds the actuation cells 201 and/or other components of the in-bed haptic device 100. The enclosure 214 may contain and/or protect the actuation cells 201 and/or other components of the in-bed haptic device 100. In some cases, the enclosure 214 is flexible. One or more surfaces of the enclosure 214 may include an adhesive or a high-friction material(s) configured to maintain the in-bed haptic device 100 in place.

The enclosure 214 may define a top external surface 216 of the in-bed haptic device 100. In various embodiments, haptic outputs provided by the in-bed haptic device 100 may be provided at and/or through the top external surface 216. In some cases, the top external surface 216 defines a modifiable contour, and actuation of the actuation cells modifies the modifiable contour to provide haptic outputs. In various embodiments, the enclosure 214 may not fully enclose or surround the components of the in-bed haptic device 100. For example, the enclosure 214 may be defined by top and bottom layers with components of the in-bed haptic device 100 in between, in which case the sides of the in-bed haptic device 100 may not be enclosed by the enclosure 214. In some cases, the enclosure 214 has an open top (e.g., the enclosure 214 does not enclose at least a portion of the top of the in-bed haptic device). For example, the array of actuation cells may define at least a portion of the top external surface 216.

The in-bed haptic device 100 may include an array of actuation cells 201 configured to expand and/or contract in predetermined sequences to provide haptic outputs. Each actuation cell 201 of the array of actuation cells may include one or more bladders defining an interior volume and configured to inflate and/or deflate to cause the actuation cells to actuate to provide haptic outputs and/or portions of haptic outputs. For example, inflation of the one or more bladders may cause the actuation cell 201 to expand, and deflation of the one or more bladders may cause the actuation cell to contract. Each bladder may be configured to inflate in response to a pressurized air (or another fluid) being introduced into the interior volume and/or deflate in response to a pressurized air being removed from the interior volume. In some cases, each actuation cell 201 of the array of actuation cells is configured to expand in a direction that is substantially transverse to the top external surface 216, thereby increasing a thickness of a region of the in-bed haptic device 100 corresponding to the cell 201. The in-bed haptic device 100 is shown in FIG. 2A as having twenty-one actuation cells 201 ordered in a two-dimensional array. This is an example and is not meant to be limiting. The in-bed haptic devices described herein may include any suitable number of actuation cells arranged in any suitable way.

As noted above, each actuation cell 201 may be individually addressed. In some cases, each actuation cell 201 may be controlled independently of all other actuation cells of the array of actuation cells. For example, providing a haptic output may include inflating a first actuation cell 201 while maintaining an adjacent actuation cell 201 in an uninflated state. In some cases, actuation cells 201 may be grouped into cell groups, and the actuation cells in the cell group are controlled together, but independently of other cell groups and/or actuation cells. To facilitate the independent control of the actuation cells 201, each actuation cell may be independently fluidly coupled (or capable of being fluidly coupled) to the control system 150. For example, each actuation cell 201 may be fluidly coupled to the control system 150 by one or more fluid paths defined in the in-bed haptic device 100, the connector 112, and/or the control system 150. In some cases, as discussed in more detail below with respect to FIG. 4, one or more valves may be positioned along the fluid path between each actuation cell 201 and the control system 150 to control the fluid coupling.

The actuation cells 201 may cause deformation and/or displacement of the top external surface 216 to provide haptic outputs and/or portions thereof. Actuation of a particular actuation cell 201 may cause deformation and/or displacement of a corresponding portion of the top external surface 216. For example, an actuation cell 201 may inflate (partially or fully) to provide a first portion of a haptic output (e.g., a first localized impulse) and may deflate (partially or fully) to provide a second portion of a haptic output (e.g., a first localized impulse). In addition, an actuation cell 201 may remain static (e.g., deflated, partially inflated, or fully inflated) during a haptic output (e.g., between inflation or deflation or while one or more other actuation cells inflate or deflate).

As noted above, as used herein, the term “haptic output” may be used to refer to a device output that is tactilely perceptible along the user's body as a series of localized impulses that are generally dynamic in nature, and the term “localized impulse” may be used to refer to a brief force acting along a portion of a user's body. A haptic output or a portion thereof may be provided by an actuation (e.g., an inflation or deflation) of one or more actuation cells 201. In some cases, the duration of an actuation of an actuation cell 201, such as an inflation period (e.g., a duration that an actuation cell is inflating) or a deflation period (e.g., a duration that an actuation cell is deflating) may be sufficiently short in duration such that the inflation and/or deflation is perceived by a user as a localized impulse. In some cases, the duration of the actuation is less than about 0.5 seconds. In some cases, the duration of the actuation is less than about one second. In some cases, the duration of the actuation is less than about five seconds.

In some cases, the duration of an actuation may be relatively long (e.g., greater than about five seconds, or greater than about 10 seconds). Similarly, a static period (e.g., a duration that an actuation cell 201 is not inflating or deflating) may be relatively short (e.g., less than about 0.5 seconds, less than about one second, less than about five seconds) or relatively long (greater than about five seconds, greater than about 10 seconds). The lengths of inflation periods, deflation periods, and static periods may be varied to provide varying haptic outputs or portions of haptic outputs. For example, a relatively short inflation period, deflation period, and/or static period may be perceived as a higher-energy pulse or a tap, while a relatively long inflation period, deflation period, and/or static period may be perceived as a lower-energy output.

In some cases, the haptic outputs include localized haptic outputs produced by one or more actuation cells 201, in which a portion of the top external surface 216 is locally displaced (e.g., moved) and/or deformed (e.g., changed in shape) relative to other portions of the top external surface. Localized haptic outputs may simulate a pulse or a tap. In some cases, the haptic outputs include global haptic outputs in which many actuation cells 201 cooperate to displace and/or deform all or substantially all (e.g., greater than 75%) of the top external surface.

In some cases, multiple actuation cells 201 may cooperate to produce a haptic output. Multiple portions of the top external surface 216 may be displaced and/or deformed by actuation of multiple different actuation cells 201 to produce a haptic output. In some cases, multiple different portions of the top external surface 216 are displaced and/or deformed in different manners according to a pattern to provide a haptic output. In some cases, actuation of the actuation cells 201 in a predetermined sequence may cause the external surface 216 to displace and/or deform according to an actual or simulated randomized pattern (e.g., no ordered pattern is discernable). In some cases, for example, the actual or simulated randomized pattern may simulate a pattern of falling raindrops. For example, a first group of one or more actuation cells 201 may inflate (partially or fully) at a first time or part of a predetermined sequence as shown in FIG. 2B, and a second group of one or more actuation cells 201 may inflate (partially or fully) at a second time or part of a predetermined sequence as shown in FIG. 2C. Different actuation cells 201 of the array of actuation cells may have inflation periods, deflation periods, and/or static periods having different lengths during a haptic output. Similarly, the same actuation cell 201 may have inflation periods, deflation periods, and or static periods having different lengths during a haptic output. In some cases, the inflation periods, deflation periods, and/or static periods of an actuation cell 201 may overlap in time with the inflation periods, deflation periods, and/or static periods of other actuation cells during a haptic output.

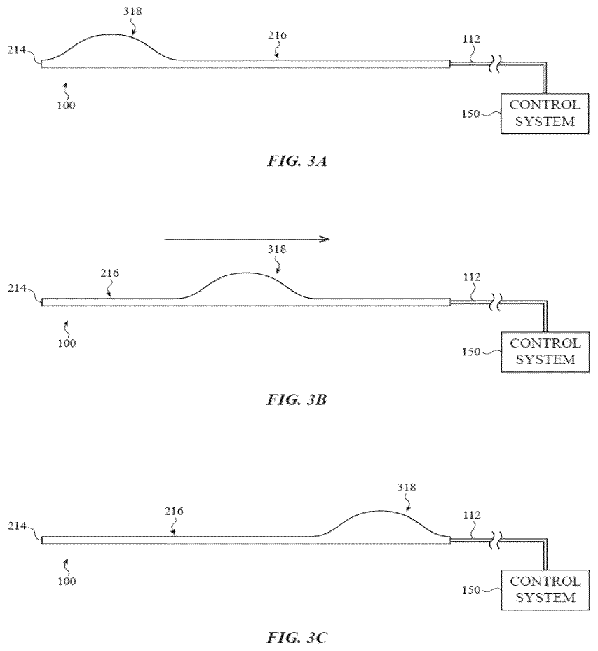

In some cases, actuation of the actuation cells 201 in a predetermined sequence may cause the external surface 216 to displace and/or deform according to an ordered (e.g., non-random) pattern. Multiple actuation cells 201 may cooperate to displace and/or deform the external surface 216 according to an ordered pattern. For example, as shown in FIGS. 3A-3C, the ordered pattern may cause a feature 318 formed by one or more actuation cells 201 to move along the external surface 216. In some cases, for example, the ordered pattern may simulate a wave or other feature moving at least partially across the external surface 216 of the in-bed haptic device 100.

The in-bed haptic device 100, the control system 150, and/or another device that includes and/or is operably connected to a processing unit may include one or more input devices (e.g., contact sensors, force sensors, audio sensors, biometric sensors, image sensors, light sensors, and the like) configured to detect inputs that are used by the processing unit to determine to provide haptic outputs and/or the types of haptic outputs. The processing unit may determine a haptic output to provide in response to one or more detected inputs and may control various components of the control system 150 and/or the in-bed haptic device 100 (e.g., valves, pumps, etc.) to provide the haptic output. Input devices and detected inputs are discussed in more detail below with respect to FIG. 4.

A haptic system may include an in-bed haptic device that is fluidly coupled to a control system that is configured to introduce pressurized air into the actuation cells and/or remove pressurized air from the actuation cells. FIG. 4 shows an example block diagram of a haptic system that includes a control system 450 that is fluidly coupled to an in-bed haptic device 400. The control system 450 and the in-bed haptic device 400 may be similar to the control system 150 and the in-bed haptic device described above and may include similar structure and/or functionality.

The control system 450 may include a fluid control system 454, which may include a high pressure reservoir, a vacuum reservoir, pumps, and/or other components used to deliver and/or remove air from the in-bed haptic device 400. The control system 450 may also include a valve array 452, pressure sensors 456 and a control unit 458. The in-bed haptic device 400 may include an array of actuation cells 401a, 401b, 401c, 401d that are configured to actuate (e.g., expand, contract, or otherwise change shape) in a predetermined sequence to provide haptic outputs. In some cases, each actuation cell 401 includes one or more bladders configured to inflate and/or deflate to actuate the actuation cells.

The fluid control system 454 may include one or more reservoirs configured to facilitate rapid inflation and/or deflation of bladders of the actuation cells 401. In some cases, the control system 450 includes one or more high pressure reservoirs containing air (or another fluid) having a pressure that is higher than atmospheric pressure and/or one or more vacuum reservoirs containing air (or another fluid) having a pressure that is lower than atmospheric pressure. The reservoirs may be any suitable containers having a fixed or variable volume, such as a tank, a bladder, or the like. The reservoirs may be formed of any suitable material(s), including polymers (e.g., PVC, polyurethane, NOMEX, HYPALON, thermoplastic, polyethylene, polyimide, cellulose, etc.), rubber, synthetic rubber, metal (e.g., aluminum, copper, etc.), fiber reinforced materials, composite materials, and the like.

The fluid control system 454 may include one or more pumps configured to establish and maintain the pressure(s) of the reservoirs. The fluid control system 454 may include a pressurizing pump configured to increase the pressure and/or maintain the increased pressure in the high pressure reservoir. The control system may include a vacuum pump configured to decrease the pressure and/or maintain the decreased pressure in the vacuum reservoir. In some cases, the fluid control system 454 may use pressure measurements of the actuation cells 401 from the pressure sensors 456. For example, the control unit 458 may receive pressure measurements for one or more of the actuation cells 401 from the pressure sensors 456 and control the valve array 452 and/or the fluid system 454 based on the measured pressures.

Using the reservoirs for inflating and/or deflating the bladders of individual actuation cells 401 of the in-bed haptic device 400 may allow the individual cells to be inflated and/or deflated more rapidly than using pumps to inflate and/or deflate the bladders. The pumps of the control system 450 can pressurize or depressurize the reservoirs over a long period of time in advance of providing haptic outputs to “charge” the reservoirs so that more rapid pressure changes may occur. The pumps may be sufficiently quiet that they do not disturb users while sleeping. For example, the pumps may include scroll-type, or rotary-type, or diffusion-type compressors or pumps.

The in-bed haptic device 400 may be fluidly coupled to the control system 450 by one or more connectors 410 (one of which is labeled for clarity). In some cases, each connector 410 connects the fluid control system 454 of the control system 450 to a respective actuation cell 401a, 401b, 401c, 401d. As noted above, each actuation cell 401 may be individually addressed. To facilitate the independent control of the actuation cells 401, each actuation cell may be independently fluidly coupled (or capable of being fluidly coupled) to the control system 450. For example, each actuation cell 401 may be fluidly coupled to the control system 450 by one or more fluid paths defined by the in-bed haptic device 400, the connectors 410, and/or the control system 450. Each of connectors 410 may define at least a portion of a fluid path between the fluid control system 454 and the bladders of a respective actuation cell 401a, 401b, 401c, 401d.

The control system 450 may include one or more valves (e.g., a valve array 452) configured to control the fluid coupling between each actuation cell 401a, 401b, 401c, 401d (e.g., the bladder(s) of each actuation cell 401) and the fluid control system 454. A valve of the valve array 452 may be opened to fluidly couple the fluid control system 454 via one or more connectors so that fluid may flow between the bladders and the reservoir. Similarly, a valve of the valve array 452 may be closed to terminate a fluid coupling so that fluid may not flow between the bladders and the fluid control system 454. The control unit 458 of the control system 450 may cause a valve between a bladder of an actuation cell 401 and the high pressure reservoir to open to inflate the bladder. Similarly, the control unit 458 may cause a valve between a bladder of an actuation cell 401 and the vacuum reservoir to deflate the bladder. In some cases, the valves may be used to modulate the flow between actuation cells 401 and a reservoir. For example, a flow may be decreased or increased using a valve.

The valves (e.g., the valve array 452) may be positioned at any suitable location along the fluid path between a reservoir and one or more bladders, including within the control system 450, connector(s) 410, in-bed haptic device 400, or actuation cells 401. The valves of the valve array 452 may be operably coupled to the control unit 458 by a connector. The valve array may include one or more motors, servos, or the like to control (e.g., open, close) the valves in response to signals received from the control unit 458. The valves of the valve array 452 may be any suitable type of valves, including ball valves, butterfly valves, choke valves, diaphragm or membrane valves, gate valves, globe valves, knife valves, needle valves, pinch valves, piston valves, plug wave valves, solenoid valves, spool valves, or the like.

The in-bed haptic device 400 may be operably coupled to the control unit 458 by a connector. The control unit 458 may receive signals (e.g., sensor signals, etc.) from the in-bed haptic device 400 and provide signals (e.g., valve control signals, etc.) to the in-bed haptic device. In some cases, the control unit determines to provide a haptic output and/or a type of haptic output to provide in response to signals received from the in-bed haptic device 400. The control unit 458 may be positioned within and/or be a component of the in-bed haptic device 400, the control system 450, or another device (e.g., a companion device).

A user input device 460 may communicably couple the control system 450 via any suitable wireless or wired communication technique. The user input device 460 may send control signals to the control unit 458 and receive signals (e.g., sensor signals) and/or other information about the control system 450 and/or the haptic device 400 (e.g., control state information, information about a current state of the haptic device 400, and so on). The user input device 460 may send signals that define one or more haptic sequences for the haptic device 400 and the control unit 458 may convert the haptic sequences into control signals that are used to control actuation of the haptic device 400.

The in-bed haptic device 400, the control system 450, and/or another device that includes and/or is operably connected to the user input device 460 may include one or more input devices (e.g., contact sensors, force sensors, audio sensors, biometric sensors, images sensors, light sensors, and the like) configured to detect inputs that are used by the processing unit to determine to provide haptic outputs and/or the types of haptic outputs to provide. The user input device 460 may determine a haptic output to provide in response to one or more detected inputs and may control various components of the control system 450 and/or the in-bed haptic device 400 (e.g., valves, pumps, etc.) to provide the haptic output.

In some cases, the input devices include one or more force sensing mechanisms for detecting input signals for use in providing haptic outputs. The force sensing mechanisms may be capable of detecting whether a user is in bed, a positioning of the user in bed, heart information, breathing information, and the like. The force sensing mechanisms may include capacitive sensing mechanisms, piezoelectric sensing mechanisms, and the like. In some cases, the input devices include one or more contact sensing mechanisms (e.g., touch and/or proximity sensing mechanisms) for detecting input signals for use in providing haptic outputs. The contact sensing mechanism may be capable of detecting whether a user is in bed, for example by detecting that the user is contacting the bed and/or the in-bed haptic device 400. The contact sensing mechanism may additionally be capable of detecting a positioning of the user in bed, (e.g., whether the user is sleeping on his or her back, side, or stomach, a relative positioning of the user in the bed, or the like). The contact sensing mechanisms and/or force sensing mechanisms may use mutual-capacitive sensing techniques and/or self-capacitive sensing techniques. The contact sensing mechanisms and/or force sensing mechanisms may include a substrate and capacitive, piezoelectric, and/or other sensing mechanisms that include one or more electrodes for determining whether a user is in contact with, proximate to, and/or exerting a force on the in-bed haptic device 400 or another device.

In some cases, the input devices include a microphone for detecting audio inputs. In some cases, the audio inputs may be used to detect snoring or other audio data as the in-bed haptic device 400 is used.

The inputs received by the control unit 458 and/or the user input device 460 may be used to determine triggers for providing haptic outputs. Triggers may indicate that a haptic output is to be produced and/or characteristics of the haptic output. In response to detecting or determining one or more triggers, the control unit 458 and/or the user input device 460 may determine one or more haptic outputs to be provided and cause the control system 450 and/or the in-bed haptic device 400 to provide the haptic output(s).

FIG. 5 shows an example process 500 for operating a haptic system such as the haptic systems described herein. The process 500 can be performed by the haptic systems described herein and include an input device (e.g., user input device 460), a control system (e.g., control system 450) and a haptic device (e.g., haptic device 400).

At operation 502, the process 500 can include displaying on, a user input device, a user interface for selecting a haptic sequence on an electronic device. In some cases, the user interface can indicate a set of haptic sequences that can be applied to the array of actuation cells, which may be predefined actuation sequences and/or user defined and saved actuation sequences. The actuation sequences may each be associated with a unique actuation control sequence for an array of actuation cells. In some cases, each of the displayed set of haptic actuation sequences can include user interface elements that indicate an effect of the actuation sequence. For example, a wave patterned haptic actuation sequence may be named to indicate the wave patterned sequence (e.g., “wave”) and/or include graphics or dynamic animations that indicate the wave pattern.

In other cases, the user interface may include controls for defining a haptic actuation sequence for an array of actuation cells. The user interface may display a graphic corresponding to the array or actuation cells. A user can interact with the displayed graphic, via a touchscreen or other suitable controls, to define a haptic output sequence that can be performed by the array of actuation cells. In some cases, the displayed graphic may include an image of the actuation pad and include images of the actuation cells of the array of actuation cells. A user may select specific cells that are to be actuated and/or a timing of the actuation. For example, the sequence that a user interacts with the displayed array of cells may be used to generate the actuation sequence. Additionally or alternatively, the displayed controls may include controls for setting the speed of the actuation, intensity of the haptic response (e.g., pressure in actuation cells), durations, and so on.

Additionally, the controls may include controls for defining a criteria for applying a defined haptic actuation sequence. The criteria may be used by the haptic system to determine when to apply a haptic actuation sequence, how long the sequence should be applied, when to transition to a different haptic sequence, and so on. For example, a user may define a first haptic actuation sequence that is applied while the user is sleeping and a second haptic actuation sequence that can be used to wake the user. The haptic actuation system may include sensors that are used to determine when a user is sleeping and apply the first haptic actuation sequence during the sleep period. A second criteria may define a wake time, or other conditions that trigger a wake sequence. In response to the second criteria being satisfied, the system may apply the second haptic actuation sequence, for example, to wake the user. The sleep and wake actuation sequences are provided as examples, and the system can apply an actuation sequence in response to other criteria such as an alert or message at an electronic device, to help a user relax in response to detecting that the user is restless, and so on.

The user interface may include controls for selecting a portion of the array to apply a haptic actuation sequence. For example, a user may select or define a haptic actuation sequence, such as a wave pattern, and then select a portion of the array of actuation cells to apply the haptic actuation sequence. In other cases, the user interface may include controls for selecting anatomical features to apply a haptic actuation sequence. For example, the haptic system can be configured to identify one or more anatomical features using pressure sensors to determine a pressure map of a user positioned on the haptic device. The user interface may display the identified anatomical features (e.g., profile of the user's torso) overlayed on the haptic array and a user may select regions of the array corresponding to specific anatomical regions. Accordingly, the haptic actuation sequence can be applied to specific anatomical features of a user. As a user moves, the system may update the pressure map to determine an updated location of the user on the bed and adjust the application of the haptic actuation sequence accordingly.

Additionally or alternatively, a user can program a haptic actuation sequence using an illustration of a torso, body, and/or body parts. The system can sense where the user's body and/or anatomical features are positioned in relation to the actuation array and apply the programmed haptic sequence to the user based on the sensed location of the user's body.

In some cases, the controls may include options for defining other parameters as an alternative and/or in addition to controlling actuation of the array. For example, the haptic system can be configured to control temperature in the haptic device using heaters and/or coolers that are integrated into a pneumatic pad placed on the bed. The controls can allow a user to define heating or cooling of the pad.

At operation 504, the process 500 can include generating a haptic control sequence based on the user input to the user interface. The haptic control sequence may be generated by a user input device and sent to the haptic control system. The haptic control sequence may be used to generate a time-varying pressure profile for actuation cells of the array of actuation cells. The time-varying pressure profile may indicate a relative and/or absolute time that each actuation cell should be actuated to achieve the defined haptic actuation sequence. Additionally the time-varying pressure profile may indicate an actuation speed, an actuation amount, an actuation duration, a deflation speed and so on. These parameters may be used by the haptic control system to generate control signals for the haptic device (e.g., valve array). For example, the control system may use the actuation speed to control valves for the actuation cells, use the actuation amount to determine a target pressure for individual actuation cells, and so on. Accordingly, the haptic control sequence can be used by the control system to determine the actuation parameters for actuation cells of the array.

At operation 506, the process 500 can include receiving sensor data at the haptic control system. The haptic control system can use sensor data to monitor and control the actuation parameters for the actuation cells. For example, pressure sensors can be used to measure pressures in the actuation cells and output the pressure measurements to the control system. The pressure measurements may be used to control the valve array and/or other components of the system to achieve the defined haptic control sequence.

The system can operate in an open loop control mode, which can include calibrating the system at one or more times and then controlling actuation of the actuation cells using parameters from the calibration process. In some cases, the system may be able to operate in an open loop control state or a closed loop control state. The pressure sensors or other sensors can be used to perform calibrations for the open loop control. For example, a calibration procedure may include determining fluid injection parameters using measured pressure data. Once calibrated, the open loop control process may include actuating cells using the determined fluid injection parameters.

Additionally or alternatively, the haptic actuation system can include temperature sensors that monitor temperature at locations along the haptic pad. The temperature measurements can be used to control heating and/or cooling of the pad. In other examples, sensors can include force, strain, capacitive, piezoelectric, and/or other suitable sensors that can be used to monitor physiological parameters of a user such as location on a pad, cardiac parameters (e.g., heart rate), respiratory parameters (e.g., breathing rate), movement of the user, and so on.

At operation 508, the process 500 can include actuating the haptic device using the haptic control sequence and sensor data. For example, the haptic control system may generate initial control signals based on the haptic control sequence and initiate actuation of the actuation cells using the initial control signals. The control system may receive sensor signals (e.g., pressure measurements) and adjust the control signals to achieve the target haptic control sequence. For example, fluid pressure applied to actuation cells may be adjusted based on a location of a user, the weight of the user, and/or other parameters. These factors may be reflected in the sensor measurements and used to adjust actuation parameters of individual actuation cells.

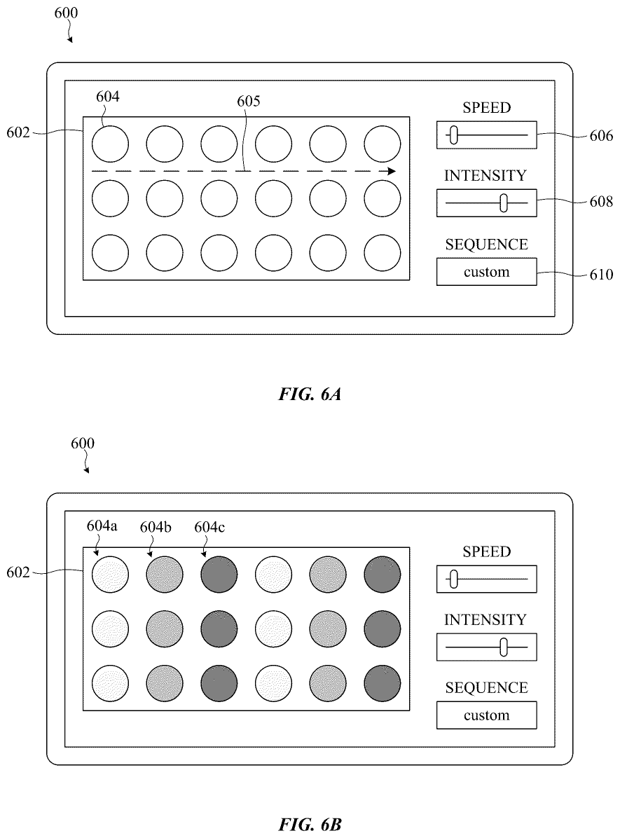

FIGS. 6A and 6B show an example user interface 600 for defining and/or selecting a haptic actuation sequence to be applied by the actuation cells. The user interface 600 may be displayed on a user input device that includes a touch-sensitive display or other input controls for receiving user inputs.

FIG. 6A shows an example of controls for defining a haptic output sequence at an array of actuation cells on a pneumatic pad. The user interface 600 can include an array graphic 602 corresponding to the array of actuation cells. The array graphic 602 can include actuation cells 604 (one of which is labeled for clarity) that are organized according to the location of the actuation cells on a pneumatic pad. For example, the layout of the actuation cells 604 in the user interface can mimic the layout of the actuation cells on the pneumatic pad and can include relative sizes and positions that reflect the relative size and position of the actuation cells. A user may be able to use the touch sensitive display to interact with the array graphic 602 to define an actuation sequence. For example, a user may be able to drag their finger across the touch sensitive display in a direction 605 to define a direction of actuation.

The timing and direction of the user input may be used to define an actuation sequence. For example, a user moving their finger in the direction 605 can be used to indicate timing of the actuation cells, which may correspond to a wave pattern across the array. That is, the actuation cells 604a may be touched first, followed by actuation cells 604b, followed by actuation cells 604c and so in FIG. 6B. The user input device may translate this interaction to a wave pattern where the actuation cells 604a are actuated first, followed by actuating the actuation cells 604b, followed by actuating the actuation cells 604c. The actuation of the cells may include both an inflation period, where fluid is introduced into the cell, followed by a deflation period, where fluid is removed from the cell.

In response to the user input, the user input device may update the user interface 600 to indicate the defined haptic actuation sequence. FIG. 6B shows an example of an actuation sequence indicated on the user interface 600. For example, the user input across the screen in the direction 605 may result in a wave patterned haptic actuation sequence in direction 605. The wave pattern may be indicated using graphic elements, which can include shading as shown in FIG. 6B. The graphical elements may display a first set of actuation cells 604a using a first shade/color to indicate a relative degree of actuation compared to other actuation cells. For example, the first set of actuation cells 604a may include lighter shading indicating a low amount or no actuation, a second set of actuation cells can include darker shading indicating a higher degree of actuation as compared to the first set of actuation cells 604a, and a third set of actuation cells 604c can include even darker shading indicating a higher degree of actuation as compared to the second set of actuation cells 604b. The degree of actuation may correspond to a pressure in the actuation cells 604 at a given point of time to indicate the dynamically changing actuation of different sets of cells.

Additionally or alternatively, the defined haptic actuation sequence may be indicated using dynamic elements such as an animation that indicates the timing of the actuation. For example, the animation for the wave pattern described above can include causing the shading/colors of the displayed actuation cells 604 to lighten and darken indicating the dynamically increasing and decreasing actuation states of the different actuation cells.

The user interface 600 can also include additional controls such as the speed control 606, the intensity control 608 and/or the sequence control 610 shown in FIG. 6A. The controls may be used to adjust aspects of the haptic actuation sequence that was input by the user. For example, the user may interact with the array graphic to define the sequence, which defines the relative time variation of actitation cells with respect to each other. The controls may be used to adjust the actuation parameters of one or more of the cells. For example, the speed control 606 may allow a user to define a speed of the actuation sequence, portions of the actuation sequence and/or actuation speed of individual actuation cells. The intensity control 608 may correspond to a degree of actuation, which in the case of a pneumatic actuation pad, can be translated into pressures for each actuation cell.

The sequence control 610 can allow a user to select a haptic actuation sequence from a set of actuation sequences, which may include predefined sequences based on previous input from the user or default sequences. In response to a user selecting a predefined sequence, the user interface 600 may display an indication (e.g., graphic or animation) of the sequence in the array graphic 602. The user may be able to interact with the user interface 600 to modify, change, or otherwise update the predefined sequence.

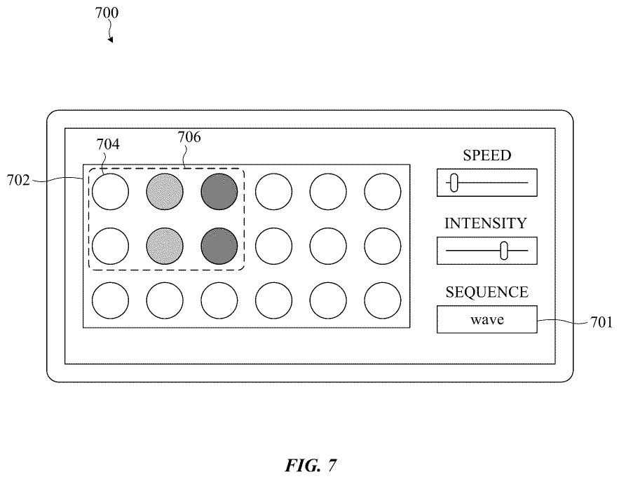

FIG. 7 shows an example user interface 700 for selecting a region of an actuation array for applying a haptic actuation sequence. The user interface 700 can include an array graphic 702 that displays actuation cells 704 of the array (e.g., as described with reference to FIGS. 6A and 6B). In some cases, a user may select a portion 706 of the array to apply a selected or defined haptic actuation sequence. For example, the user may interact with the user interface 700 to select portion 706. In response, the input device may define a haptic actuation control system that causes actuation of the sequence on the corresponding portion of the array of actuation cells. In some cases, the unselected portions may remain inactive during the actuation sequence. In other cases, a user can define different actuation sequences for different portions of the array. For example, a user may interact with the user interface 700 to define a first actuation sequence for the portion 706 and define a second actuation sequence for a second portion of the array.

The user input device can use the inputs to the user interface 700 to generate a haptic actuation control sequence which can be used by the haptic actuation control system to actuate the array of actuation cells, as described herein.

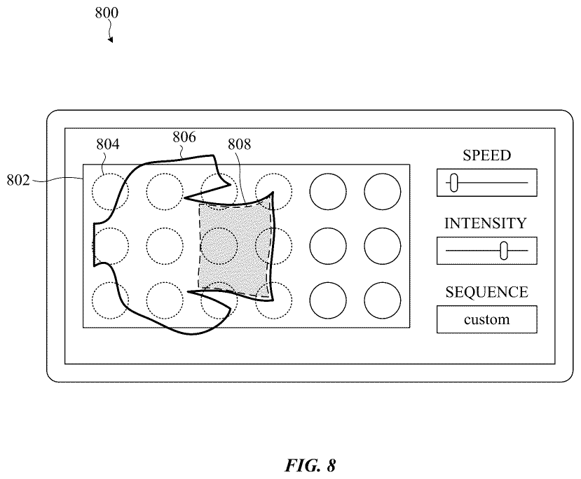

FIG. 8 shows an example user interface 800 for selecting anatomical features for applying a haptic actuation sequence. The user interface 800 can include an array graphic 802 which displays actuation cells 804 of the array (e.g., as described with reference to FIGS. 6A and 6B).

As described herein, the array of actuation cells may include one or more sensors that can be configured to identify the body position of a user or specific anatomical features of a user positioned on the pneumatic pad of the actuation array. For example, pressure sensors can measure pressures in actuation cells and the pressure measurements can be used to generate a pressure map of a user positioned on the actuation array. The pressure map can be used to identify anatomical features of a user such as the profile of a user's torso with respect to the actuation array. Additionally or alternatively other sensing modalities can be used, such as capacitive sensing, strain sensing, force sensing, temperature sensing, and so on.

The user interface 800 can display a user graphic 806, which shows a location of anatomical features of a user with respect to the array graphic 802, which can indicate the user's positioning on the actuation array. A user may interact with the user interface 800 to select anatomical features for applying a haptic actuation sequence. For example, the user input may select a region 808 of the user body. The user input device may use the selection of region 808 to generate haptic control sequences that cause the haptic output to be applied to a portion of the user corresponding to region 808.

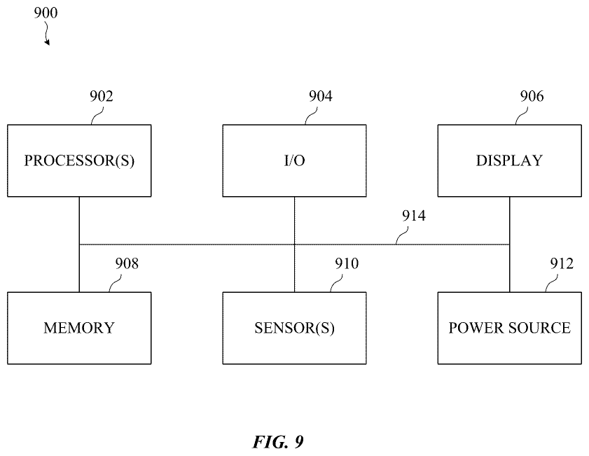

FIG. 9 is an example block diagram of a haptic system 900, which can take the form of any of the devices or collection of two or more devices that, in combination, provide various aspects of the haptic system as described with references to FIGS. 1-8. The devices 900 can include a processor 902, an input/output (I/O) mechanism 904 (e.g., wired or wireless communications interfaces), a display 906, memory 908, sensors 910 (e.g., physiological sensors such as those described herein), and a power source 912 (e.g., a rechargeable battery). The processor 902 can control some or all of the operations of the devices 900. The processor 902 can communicate, either directly or indirectly, with some or all of the components of the devices 900. For example, a system bus or other communication mechanism 914 can provide communication between the processor 902, the I/O mechanism 904, the memory 908, the sensors 910, and the power source 912.

The processor 902 can be implemented as any electronic device capable of processing, receiving, or transmitting data or instructions. For example, the processor 902 can be a microprocessor, a central processing unit (CPU), an application-specific integrated circuit (ASIC), a digital signal processor (DSP), or combinations of such devices. As described herein, the term “processor” is meant to encompass a single processor or processing unit, multiple processors, multiple processing units, or other suitable computing element or elements. The processing unit can be programmed to perform the various aspects of the systems described herein.

It should be noted that the components of the devices 900 can be controlled by multiple processors. For example, select components of the devices 900 (e.g., a sensor 910) may be controlled by a first processor and other components of the devices 900 (e.g., the I/O 904) may be controlled by a second processor, where the first and second processors may or may not be in communication with each other.

The I/O device 904 can transmit and/or receive data from a user or another electronic device. An I/O device can transmit electronic signals via a communications network, such as a wireless and/or wired network connection. Examples of wireless and wired network connections include, but are not limited to, cellular, Wi-Fi, Bluetooth, IR, and Ethernet connections. In some cases, the I/O device 904 can communicate with an external electronic device, such as a smartphone, electronic device, or other portable electronic device, as described here.

The devices 900 may optionally include a display 906 such as a liquid-crystal display (LCD), an organic light emitting diode (OLED) display, a light emitting diode (LED) display, or the like. If the display 906 is an LCD, the display 906 may also include a backlight component that can be controlled to provide variable levels of display brightness. If the display 906 is an OLED or LED type display, the brightness of the display 906 may be controlled by modifying the electrical signals that are provided to display elements. The display 906 may correspond to any of the displays shown or described herein.

The memory 908 can store electronic data that can be used by the devices 900. For example, the memory 908 can store electrical data or content such as, for example, audio and video files, documents and applications, device settings and user preferences, timing signals, control signals, and data structures or databases. The memory 908 can be configured as any type of memory. By way of example only, the memory 908 can be implemented as random access memory, read-only memory, Flash memory, removable memory, other types of storage elements, or combinations of such devices.

The devices 900 may also include one or more sensors 910 positioned almost anywhere on the devices 900. The sensor(s) 910 can be configured to sense one or more types of parameters, such as but not limited to, pressure, light, touch, heat, movement, relative motion, biometric data (e.g., biological parameters), and so on. For example, the sensor(s) 910 may include a heat sensor, a position sensor, a light or optical sensor, an accelerometer, a pressure transducer, a gyroscope, a magnetometer, a health monitoring sensor, and so on. Additionally, the one or more sensors 910 can utilize any suitable sensing technology, including, but not limited to, capacitive, ultrasonic, resistive, optical, ultrasound, piezoelectric, and thermal sensing technology.

The power source 912 can be implemented with any device capable of providing energy to the devices 900. For example, the power source 912 may be one or more batteries or rechargeable batteries. Additionally or alternatively, the power source 912 can be a power connector or power cord that connects the devices 900 to another power source, such as a wall outlet.

As described above, one aspect of the present technology is directed to monitoring physiological conditions of a user to provide haptic feedback via a haptic actuation system and the like. The present disclosure contemplates that in some instances this gathered data may include personal information data that uniquely identifies or can be used to contact or locate a specific person. Such personal information data can include demographic data, location-based data, telephone numbers, email addresses, Twitter IDs (or other social media aliases or handles), home addresses, data or records relating to a user's health or level of fitness (e.g., vital signs measurements, medication information, exercise information), date of birth, or any other identifying or personal information.

The present disclosure recognizes that the use of such personal information data, in the present technology, can be used to the benefit of users. For example, the personal information data can be used to provide haptic or audiovisual outputs that are tailored to the user. Further, other uses for personal information data that benefit the user are also contemplated by the present disclosure. For instance, health and fitness data may be used to provide insights into a user's general wellness or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining the privacy and security of personal information data. Such policies should be easily accessible by users and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and revised to adhere to applicable laws and standards, including jurisdiction-specific considerations. For instance, in the US, collection of or access to certain health data may be governed by federal and/or state laws, such as the Health Insurance Portability and Accountability Act (“HIPAA”); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, in the case of determining spatial parameters, the present technology can be configured to allow users to select to “opt in” or “opt out” of participation in the collection of personal information data during registration for services or anytime thereafter. In addition to providing “opt in” and “opt out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data. For example, haptic outputs may be provided based on non-personal information data or a bare minimum amount of personal information, such as events or states at the device associated with a user, other non-personal information, or publicly available information.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. An in-bed haptic system comprising:
a pneumatic pad configured for placement between a user and a bed and comprising:
an array of actuation cells, wherein each actuation cell in the array of actuation cells is configured to actuate in response to fluid being introduced into an actuation cell; and
an array of pressure sensors, each pressure sensor in the array of pressure sensors configured to output pressure measurements corresponding to the array of actuation cells;
an input device comprising a touch sensitive display and configured to:
display a set of haptic sequences that can be applied to the array of actuation cells in a user interface of the input device; and
receive an input at the touch sensitive display of a selected haptic sequence of the set of haptic sequences; and
a control system configured to;
receive an indication of the selected haptic sequence;
receive the pressure measurements; and
actuate actuation cells of the array of actuation cells using the selected haptic sequence and the pressure measurements.

2. The in-bed haptic system of claim 1, wherein:
each pressure sensor in the array of pressure sensors is configured to output the pressure measurements corresponding to an actuation cell in the array of actuation cells;
the selected haptic sequence defines a pressure profile for actuation cells in the array of actuation cells; and
the control system is configured to actuate the actuation cells in accordance with the pressure profile corresponding to each actuation cell using the pressure measurements corresponding to each actuation cell.

3. The in-bed haptic system of claim 1, wherein:
the set of haptic sequences each defines a time-varying pressure profile for the array of actuation cells; and
displaying the set of haptic sequences comprises displaying a dynamic animation for each haptic sequence in the set of haptic sequences, the dynamic animation indicating the time-varying pressure profile that can be applied to the array of actuation cells.

4. The in-bed haptic system of claim 1, wherein:
one or more haptic sequences in the set of haptic sequences define a temperature profile for the array of actuation cells; and
the temperature profile causes the control system to change a temperature of one or more portions of the array of actuation cells during the actuation of the actuation cells.

5. The in-bed haptic system of claim 1, wherein:
receiving the input of the selected haptic sequence comprises receiving a selection of a portion of the array of actuation cells to apply the selected haptic sequence; and
in response to receiving the selected haptic sequence, the control system is configured to apply the selected haptic sequence to the selected portion of the array of actuation cells.

6. The in-bed haptic system of claim 5, wherein:
the input device is configured to display a graphic corresponding to the array of actuation cells; and
the selection of the portion of the array of actuation cells comprises a user input to the touch sensitive display selecting the portion of the array of actuation cells from the graphic.

7. The in-bed haptic system of claim 1, wherein:
a set of haptic patterns comprises an option for inputting a custom haptic actuation sequence;
in response to the user inputting the custom haptic actuation sequence, the input device is configured to generate a user defined haptic sequence; and
in response to receiving the user defined haptic sequence, the control system is configured to actuate the array of actuation cells in accordance with the user defined haptic sequence.

8. The in-bed haptic system of claim 7, wherein:
in response to a user selection of the option for inputting the custom haptic actuation sequence, the input device is configured to display a graphic corresponding to the array of actuation cells; and
the custom haptic actuation sequence is determined from a user input to the displayed graphic.

9. A pneumatic actuation device comprising:
a pneumatic pad configured for placement between a user and a bed and comprising an array of actuation cells, wherein each actuation cell in the array of actuation cells is configured to actuate in response to fluid being introduced into an actuation cell; and
a control system configured to;
receive a haptic control sequence from a user input to an input device, the haptic control sequence generated using the user input to a user interface comprising one or more controls for defining a haptic output at the pneumatic pad; and
actuate the array of actuation cells using the haptic control sequence.

10. The pneumatic actuation device of claim 9, wherein:
the haptic control sequence indicates an actuation profile for the array of actuation cells; and
the control system determines a time-varying pressure profile for actuation cells in the array of actuation cells based on the actuation profile.

11. The pneumatic actuation device of claim 10, wherein:
the pneumatic pad further comprises an array of pressure sensors, each pressure sensor in the array of pressure sensors configured to output pressure measurements corresponding to the actuation cell in the array of actuation cells; and
the control system is configured:
to receive the pressure measurements from the array of pressure sensors; and
actuate actuation cells using the time-varying pressure profile and the received pressure measurements.

12. The pneumatic actuation device of claim 10, wherein:
the user input comprises a user interaction with a graphic corresponding to the array of actuation cells; and
the haptic control sequence is generated using the user interaction with the graphic.

13. The pneumatic actuation device of claim 9, wherein:
the haptic control sequence defines one or more temperature profiles for the array of actuation cells; and
the control system is configured to change a temperature in actuation cells of the array of actuation cells in accordance with the one or more temperature profiles.

14. The pneumatic actuation device of claim 9, wherein:
the haptic control sequence is a first haptic control sequence that comprises a first set of criteria for applying the first haptic control sequence to the array of actuation cells; and
the control system is configured to:
receive a second haptic control sequence that defines a second haptic output and a second set of criteria for applying the second haptic output.

15. The pneumatic actuation device of claim 14, wherein:
in response to the first set of criteria being satisfied, the control system is configured to actuate the array of actuation cells in accordance with the first haptic control sequence; and
in response to the second set of criteria being satisfied, the control system in configured to actuate the array of actuation cells in accordance with the second haptic control sequence.

16. The pneumatic actuation device of claim 15, wherein:
the first set of criteria defines a sleep period for the user; and
the second set of criteria defines a wake period for the user.

17. An in-bed haptic system comprising:
a pneumatic pad configured for placement between a user and a bed and comprising an array of inflatable cells;
an input device configured to:
display a user interface for defining a haptic output; and
in response to receiving an input to the user interface, generate a haptic control sequence for applying to the array of inflatable cells; and
a control system configured to;
receive the haptic control sequence; and
actuate the pneumatic pad using the haptic control sequence.

18. The in-bed haptic system of claim 17, wherein:
the input defines a region of the pneumatic pad for applying the haptic control sequence; and
the control system is configured to actuate the array of inflatable cells in the region of the pneumatic pad.

19. The in-bed haptic system of claim 17, wherein:
the pneumatic pad comprises an array of pressure sensors configured to measure pressures of the array of inflatable cells; and
the control system is configured to:
receive the pressure measurements from the array of pressure sensors; and
determine a location of one or more anatomical features of the user.

20. The in-bed haptic system of claim 19, wherein:
the input defines a region of a user's body for applying the haptic control sequence; and
the control system actuates the pneumatic pad in the region of the user's body using the location of the one or more anatomical features.
